(19) United States
(12) Patent Application Publication
(10) Pub. No.: US 2004/0126325 A1
(43) Pub. Date: Jul. 1, 2004

(54) Medicinal aerosol solution formulation products with improved chemical stability

(76) Inventors: David Lewis, Wiles (GB); David Ganderton, Chertton Bishop (GB); Brian Meakin, Bath (GB); Gaetano Brambilla, Parma (IT); Alessandra Ferraris, Milano (IT)

Correspondence Address: HOLME ROBERTS & OWEN, LLP, Suite 1800, 299 South Main Street, Salt Lake City, UT, 84111, US

(21) Appl. No.: 10/657,759

(22) Filed: Sep. 8, 2003

Related U.S. Application Data
(63) Continuation of application No. PCT/EP02/02710, filed on Mar. 12, 2002.

(51) Int. Cl. A61L 9/04; A61M 11/00

(52) U.S. Cl. 424/45; 128/200.23

(57) ABSTRACT

The present invention relates to a medicinal aerosol solution formulation product with improved chemical stability, comprising a pressurized metered dose inhaler, comprising an aerosol canister equipped with a metering valve and containing a medicinal aerosol solution formulation containing an active ingredient subject to a degradation by means of peroxides or other leachables, a hydrofluorocarbon propellant, a co-solvent and optionally a low-volatility component, wherein optionally part or all of the internal surfaces of said inhaler consists of stainless steel, anodized aluminum or are lined with an inert organic coating, and wherein the canister has a rim with rounded edges which avoids contact of a sharp edge with the rubber materials used as valve gaskets.

### CROSS-REFERENCE TO RELATED APPLICATIONS

This application is a continuation of the Patent Cooperation Treaty application PCT/EP 02/02710, having an international filing date of Mar. 12, 2002, which application is incorporated herein in its entirety, and to which priority is claimed.

### BACKGROUND OF THE INVENTION

The present invention relates to medicinal aerosol products and, in particular, to aerosol products such as metered dose inhalers (MDIs) for delivery of aerosol solution formulations containing an active ingredient subject to a degradation by means of peroxides or compounds that can leach from elastomeric or plastic components and closure system as a result of direct contact with the formulation of the MDI. These compounds are defined in the following as leachables. In a preferred embodiment of the invention the active ingredient is a corticosteroid and more preferably a 20-ketosteroid. These kinds of active ingredients have been found to be highly susceptible to chemical degradation when formulated as solution aerosol products. The present invention provides a way of improving chemical stability of such steroids in aerosol solution formulations.

Pressurized metered dose inhalers are well known devices for administering pharmaceutical products to the respiratory tract by inhalation.

Active materials commonly delivered by inhalation include bronchodilators such as β2 agonists and anticholinergics, corticosteroids, anti-leukotrienes, anti-allergics and other materials that may be efficiently administered by inhalation, thus increasing the therapeutic index and reducing side effects of the active material.

MDI uses a propellant to expel droplets containing the pharmaceutical product to the respiratory tract as an aerosol.

For many years the preferred propellants used in aerosols for pharmaceutical use have been a group of chlorofluorocarbons which are commonly called Freons or CFCs, such as CCl₃F (Freon 11 or CFC-11), CCl₂F₂ (Freon 12 or CFC-12), and CClF₂-CClF₂ (Freon 114 or CFC-114).

Recently, the chlorofluorocarbon (CFC) propellants such as Freon 11 and Freon 12 have been implicated in the destruction of the ozone layer and their production is being phased out.

Hydrofluoroalkanes [(HFAs) known also as hydro-fluoro-carbons (HFCs)] contain no chlorine and are considered less destructive to ozone and these are proposed as substitutes for CFCs.

HFAs and in particular 1,1,1,2-tetrafluoroethane (HFA 134a) and 1,1,1,2,3,3,3-heptafluoropropane (HFA 227) have been acknowledged to be the best candidates for non-CFC propellants and a number of medicinal aerosol formulations using such HFA propellant systems have been disclosed.

Many of these applications, in which HFAs are used as propellant, propose the addition of one or more adjuvants including compounds acting as co-solvents, surface active agents including fluorinated and non-fluorinated surfactants, dispersing agents including alkylpolyethoxylates and stabilizers.

The co-solvent is usually an alcohol, preferably ethanol. Among the additives, a low volatility component can be present, having a vapor pressure at 25° C. lower than 0.1 kPa, preferably lower than 0.05 kPa. Advantageously, it could be selected from the group of glycols, particularly propylene glycol, polyethylene glycol and glycerol or esters, for example ascorbyl palmitate, isopropyl myristate and tocopherol esters.

The compositions of the invention may contain from 0.1 to 10% w/w of said low volatility component, preferably between 0.3 to 5% w/w, more preferably between 0.5 and 2.0% w/w.

Compositions for aerosol administration via MDIs can be solutions or suspensions. The solution type aerosol formulation contains the medicament dissolved or solubilized in the propellant, or a mixture of propellant and co-solvent. The suspension type aerosol formulation contains the medicament in the form of particles which are dispersed in the propellant. The suspension type aerosol formulations usually contain a surfactant, and can also include a co-solvent. Solution compositions offer several advantages: they are convenient to manufacture because the active ingredient can be substantially completely dissolved in the propellant vehicle and because they obviate physical stability problems associated with suspension compositions, such as increase of particle size, crystal polymorphism, flocculation, particle aggregation, all of which affect dose uniformity.

On the other hand the widespread use of solution formulations is limited by their chemical instability, causing the formation of degradation products.

The international application WO 00/30608 of the applicant relates to pressurized metered dose inhalers wherein part or all of the internal surfaces consist of stainless steel, anodized aluminum or are lined with an inert organic coating to enhance the chemical stability of active ingredients in solution in a hydrofluorocarbon propellant, a co-solvent and optionally a low volatility component.

WO 96/32099 discloses metered dose inhalers for the administration of different active ingredients in suspension in the propellant, wherein the internal surfaces of the inhaler are partially or completely coated with one or more fluorocarbon polymers optionally in combination with one or more non-fluorocarbon polymers to reduce or essentially eliminate the problem of adhesion or deposition of the active ingredient particles on the can walls and thus ensure consistent delivery of the aerosol from the MDI.

It is also known from Eur. J. Pharm. Biopharm. 1997, 44, 195 that suspensions of drugs in HFA propellant are frequently subjected to absorption of the drug particles on the valves and on the internal walls of the inhaler.

WO 95/17195 describes aerosol compositions comprising flunisolide, ethanol and HFA propellants. It is stated in the document that conventional aerosol canisters can be used to contain the composition and that certain containers enhance its chemical and physical stability. It is suggested that the composition can be preferably contained in vials coated with resins such as epoxy resins (e.g. epoxy-phenolic resins and epoxy-urea-formaldehyde resins).

The compositions are preferably dispensed via a valve assembly wherein the diaphragm is fashioned by extrusion, injection molding or compression molding from a thermoplastic material such as FLEXOMER™ GERS 1085 NT polyolefin (Union Carbide). Another suitable valve rubber is a nitrile rubber (“DB-218”) available from American Gasket and Rubber, Schiller Park. Ill.

WO 00/35458 addresses the problem of preparing a stable solution formulation of budesonide, suitable for use as an aerosol, that remains chemically and physically stable during storage at ambient conditions of temperature and humidity.

The above objectives are achieved with a formulation containing unusually high concentrations of the co-solvent. The most preferred co-solvent is ethanol and it is preferably present in an amount of at least 10% by weight, more preferably at least 15% by weight, even more preferably at least 20% by weight and most preferably at least 25% by weight.

On the other hand it is well known that high quantities of ethanol are detrimental to the formulation performance, since they cause a decrease of the fine particle dose, i.e. of the quantity of particles of the active ingredient reaching the peripheral airways.

WO 00/78286 teaches that certain steroids, particularly 20-ketosteroids, are highly susceptible to chemical degradation when formulated as solution aerosol products and stored in contact with aerosol containers made of metal, usually aluminum. The chemical degradation is particularly influenced by the metal oxide e.g. Al₂O₃ layer that forms on the interior surface of the container.

In WO 00/78286 the chemical instability of aerosol formulations containing steroids is emphasized. A great deal of research has been directed at steroid degradation. Chemical degradation is especially problematic when the steroid is dissolved in the formulation and, consequently, the vast majority of marketed MDI steroid products are formulated as particulate suspensions of the steroid, which are much less susceptible to chemical degradation than solutions. The inventors according to WO 00/78286 believe that all currently marketed CFC-containing MDI products for delivering steroids are available only as particulate suspension formulations in CFC-propellants. However, suspension formulations of a medicament are more likely to encounter problems with physical instability (e.g. agglomeration, crystal growth and deposition on the container wall, all resulting in inconsistent dosage delivery).

Furthermore, according to WO 00/78286, until now there has been no way to identify which steroids are likely to be most stable as solution aerosols and which will be most sensitive to degradation in solution aerosol products or how to reduce such degradation.

The suggested solution in WO 00/78286 is to use an aerosol container having a non-metal interior surface. It is also suggested to similarly coat the metal valve components in contact with the formulation with an inert material. The inert material is selected from epoxy-phenolic lacquer, perfluoroethylenepropylene and a very thin layer of fused silica glass.

Alumina-catalyzed degradation of corticosteroids such as budesonide or triamcinolone acetonide in ethanol solution were also presented to the American Association of Pharmaceutical Scientists—AAPS meeting held in Indianapolis , Ind. (USA) from Oct. 20 to Nov. 2, 2000.

### BRIEF SUMMARY OF THE INVENTION

The present invention relates to a medicinal aerosol solution formulation product with improved chemical stability for use in connection with a pressurized metered dose inhaler.

More specifically, an aerosol canister is equipped with a metering valve and filled with a medicinal aerosol solution formulation containing an active ingredient subject to a degradation by means of peroxides or other leachables, a hydrofluorocarbon propellant, a co-solvent and optionally a low-volatility component. It is a feature of the present invention that the canister is provided with a rim having rounded edges which avoids contact of a sharp edge with the rubbers used as valve gaskets.

These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIGS. 1A and 1B are cross-sectional views illustrating one form of metered dose inhalers according to the prior art.

FIG. 2 is a cross-sectional view of a canister of a metered dose inhaler in accordance with one aspect of the present invention

FIG. 3 is a cross-sectional view of the neck area of a canister of a metered dose inhaler in accordance with an aspect of the present invention.

FIG. 4 is a cross-sectional view of the neck area of another canister of a metered dose inhaler in accordance with an aspect of the present invention.

FIG. 5 is a cross-sectional view of the neck area of yet another canister of a metered dose inhaler in accordance with an aspect of the present invention.

### DETAILED DESCRIPTION OF THE PREFERRED EMBODIMENTS

It has now been found that the chemical degradation of steroids in solution in a HFA propellant/ethanol system can depend not only upon metal oxides of the layer of the interior surface of the container but also upon the peroxides released from the rubbers used as valve gasket or other compounds that can leach from the closure system into the formulation.

Pressurized metered dose inhalers are known devices, usually consisting of a main body or canister (can) acting as a reservoir for the aerosol formulation, a cap sealing the main body and a metering valve fitted in the cap.

MDIs are usually made of a conventional material such as aluminum, tin plate, glass, plastic and the like.

The filling of a metered dose inhaler with a composition comprises the following steps:

1. Weighing the required components (one or more active materials, one or more co-solvents, optional excipients) into a canister;

2. Crimping a valve upon the canister;

3. Adding a known amount of propellant through the valve into the canister.

It has now been found that the crimping of the valve upon the can may cause breaks in the rubbers used as valve gaskets and consequent release of peroxides and other leachables with time.

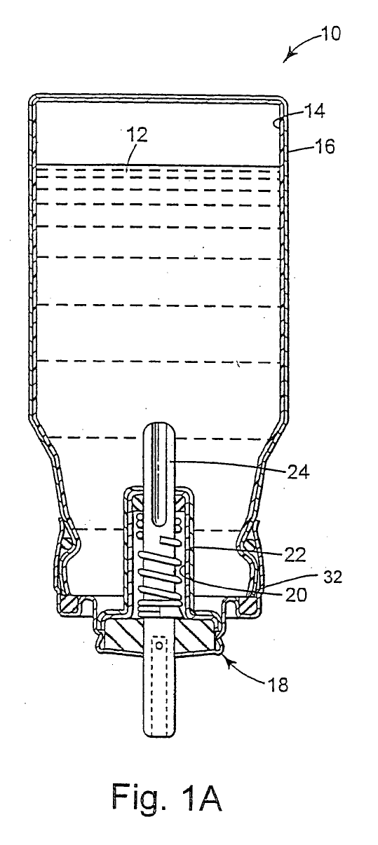

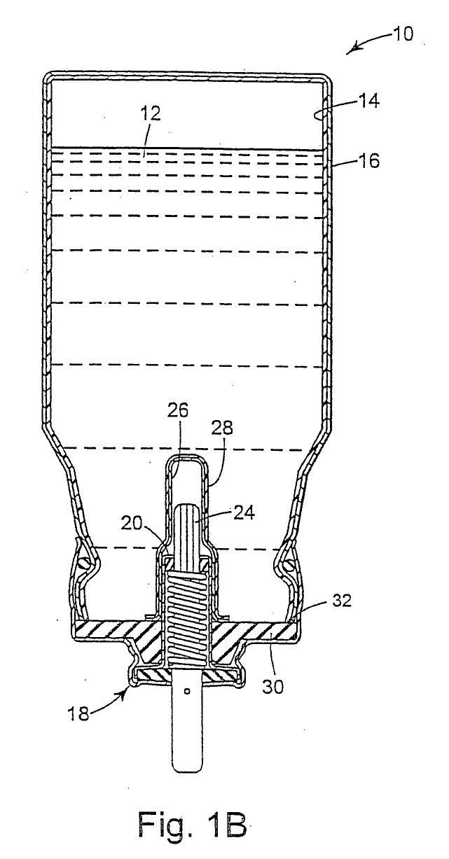

The standard canisters, in fact, have a cutting edge opening; see FIGS. 1A and 1B. FIGS. 1A and 1B show a cross-sectional view of a metered dose inhaler containing a medicinal 20-ketosteroid formulation according to the prior art (WO 00/78286).

The cutting edge during the crimping process may lead to a damage and compression of the surface of the rubber, thus facilitating the migration of components from the rubber to the solution.

The present invention provides a medicinal aerosol solution formulation product with enhanced chemical stability. Such a product consists of a main body or canister (or can) acting as a reservoir for the aerosol formulation, a cap sealing the main body and a metering valve fitted in the cap, characterized in that the canister has no cutting edge opening, i.e. it has rounded edges. Moreover, preferably the valves are washed with a suitable pharmaceutically acceptable solvent, preferably ethanol before the metered dose inhaler is built. In general, solvents which are pharmaceutically acceptable and endowed with adequate capacity of extraction of oxides and peroxides can be utilized. In the medicinal aerosol solution formulation product of the present invention breaks in the rubbers used as valve gaskets are excluded by avoiding a cutting edge opening at the rim of the canisters.

The avoidance of a cutting edge opening can be obtained by different means, i.e. by increasing the width of the material utilized for manufacturing the cans, flattening or rounding the edge of the rim with suitable means in order to avoid the presence of sharp edges, or rolling-in or over the rim.

According to the present invention, a canister having a rim with rounded edges, preferably a rolled neck, or a rolled-in rim, a part or full rollover rim is used for the preparation of aerosol solution formulation products containing an active ingredient subject to a degradation by means of peroxides or other leachables.

In a preferred embodiment of the invention the active ingredient is a corticosteroid and more preferably a 20-ketosteroid.

Preferably the pressurized metered dose inhaler (MDI) used in the present invention is one as described in applicant's earlier patent application PCT/EP99/09002 published as WO 00/30608. Accordingly, in a preferred embodiment of the present invention, part or all of the internal surfaces of the MDI consist of stainless steel, anodized aluminum or is lined with an inert organic coating. Any kind of stainless steel may be used. The preferred material of the aerosol cans is anodized aluminum. Examples for preferred inert organic coatings are perfluoroalkoxyalkane, perfluoroalkoxyalkylene, perfluoroalkylenes such as polytetrafluoroethylene, epoxy-phenol resins, fluorinated-ethylene-propylene, polyether sulfone and a copolymer fluorinated-ethylene-propylene polyether sulfone.

Other suitable coatings could be polyamide, polyimide, polyamideimide, polyphenylene sulfide or their combinations.

The invention will now be described with reference to the following drawings. FIGS. 1A and 1B show a metered dose inhaler containing a medicinal 20-ketosteroid formulation according to the prior art (WO 00/78286). FIG. 1B is the same as FIG. 1A but with a modified valve configuration.

FIG. 1A shows a medicinal aerosol device 10 comprising a pressurizable metal aerosol container 16 equipped with a metering valve 18. The metal container 16 is preferably made of aluminum (i.e. aluminum or aluminum alloy) and in this particular embodiment has an inert interior coating layer 14. The metering valve 18 includes a metal metering chamber 20 with a coating layer 22.

FIG. 1B shows an alternative embodiment that is essentially the same as FIG. 1A but utilizes a fixed bottle emptier 26 with coating layer 28. Also, a solution gasket 30 is used to further prevent contact of the formulation with metal components.

WO 00/78286 does not appreciate to avoid any breakage of the rubbers used as valve gaskets to avoid any release of leachables with time. As can be taken from FIGS. 1A and 1B, the canisters used according to WO 00/78286 have a cutting edge opening 32 in contact with the rubbers used as valve gaskets which may cause breaks in the rubbers.

Instead of a cutting edge 32 the canisters according to the present invention have a rounded edge in order to avoid any breaks in the rubbers used as valve gaskets. The rim can be of any kind avoiding contact of a sharp edge with the rubbers used as valve gaskets.

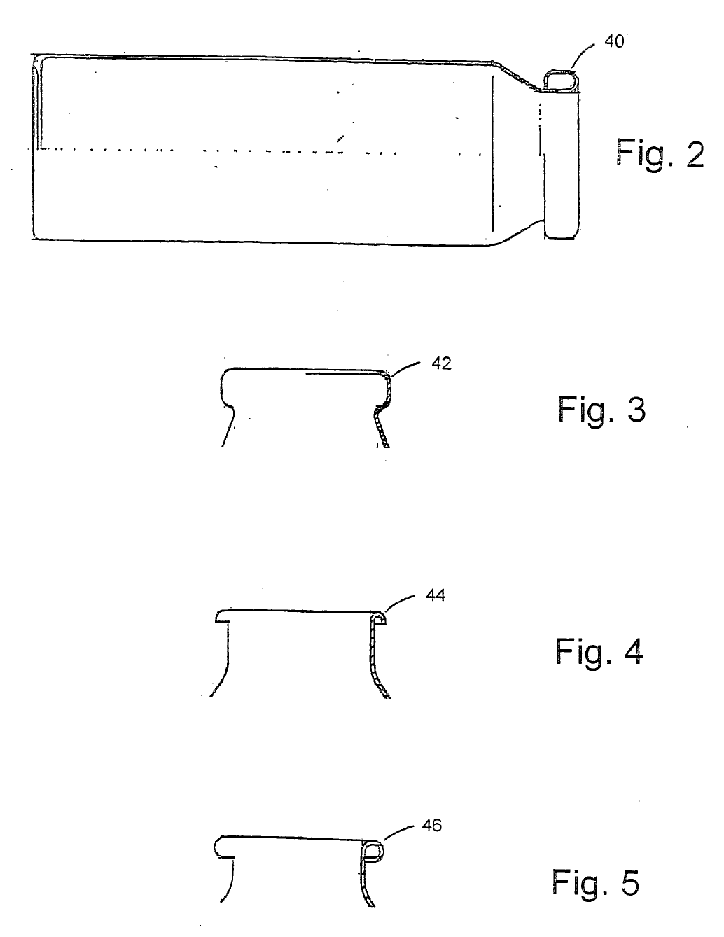

FIG. 2 is a cross-sectional view of a canister with a rolled neck 40 used in a metered dose inhaler containing a medicinal aerosol solution formulation with enhanced chemical stability according to the present invention.

FIG. 3 is a cross-sectional view of the neck of a canister with a rolled-in rim 42.

FIG. 4 is a cross-sectional view of the neck of a canister with a part rollover rim 44.

FIG. 5 is a cross-sectional view of the neck of a canister with a full rollover rim 46.

The canisters according to FIGS. 2 to 5 can be used in conventional metered dose inhalers or in metered dose inhalers as described in applicant's earlier patent application WO 00/30608 wherein part or all of the internal surfaces consist of stainless steel, anodized aluminum or are lined with an inert organic coating.

Moreover, in a particular preferred embodiment the valve gaskets are washed before crimping of the valve upon the can with ethanol in order to remove any impurities such as metal oxides, peroxides and other leachables from the rubbers used as valve gaskets and other components.

Corticosteroids are among the preferred active ingredients since it has been demonstrated that they are subject to degradation by means of peroxides. However, the metered dose inhalers of the invention apply to any active ingredient that can meet problems of chemical stability provoked by peroxides or other leachables, directly or indirectly, for example modifying the apparent pH of the solution.

In the prior art, in EP 0 673 240 medicaments are disclosed, selected from the group consisting of ipratropium bromide, oxitropium bromide, albuterol, tiotropium bromide and fenoterol, that exhibit significant degradation in the HFA propellant/co-solvent system. The degradation is pH dependent and, according to the inventors, can be solved by adding an inorganic or an organic acid.

WO 01/89480 discloses HFA aerosol solution compositions containing a P2-agonist of the phenylalkylamino class as a medicament, wherein the phenylalkylamino compound is particularly sensitive to pH variations and can be only stabilized with a strictly controlled pH range.

The examples which follow have been conducted with a solution formulation of budesonide in ethanol, glycerol and HFA 134a.

The invention naturally applies to any formulation of a steroid and especially of a 20-ketosteroid in solution in a HFA propellant which meets with problems of chemical stability.

Examples of this kind of steroids are budesonide and its epimers, mometasone furoate, triamcinolone acetonide, butixocort, ciclesonide.

It has been observed in budesonide solution formulations in HFA 134a (norflurane) and ethanol put on stability at various temperatures both up-right and inverted that notwithstanding the use of anodized aluminum or epoxy-phenol lacquered cans at high temperature the formulations are chemically stable for the first months, after which an acceleration of the oxidation process takes place.

The chemical degradation is slowed when valves extracted (washed) with ethanol are employed. In fact the washing removes the impurities and among these also peroxides and possible metal oxides surrounding the metallic part of the valve.

### EXAMPLE 1

Cans of Budesonide in solution according to the following formulation were manufactured:

Budesonide 400 mg (200 mcg/shot)

Glycerol 1.3% (w/w)

Ethanol 15.0% (w/w)

Norflurane q.b. a 100 ml

Anodized and lacquered cans and non ethanol extracted valves were utilized. The cans were put on stability at 50° C. (inverted)

A high degree of degradation was observed.

TABLE 1
Stability of Budesonide solution formulations (inverted)
Recovery (%) of budesonide after storage at different times and
temperatures (average values referred to two tests)

| Can Type | | | |
|---|---|---|---|
| (cut edge | | | |
| can) | t = 0 | t = 2 months T = 50° C. | t = 5 months T = 50° C. |
| Anodized | 99.42% | — | 33.38% |
| Aluminum | | | |
| Lacquered | 99.66% | — | 36.86% |

t = time
T = temperature

The degradation is an oxidative one and it was explained with the presence of peroxides in the rubbers.

### EXAMPLE 2

Cans with the same formulation were prepared and in this case ethanol extracted valves were utilized. During the ethanol extraction the surface of the valves was washed of the peroxides and the solution formulation exhibits a much higher stability.

TABLE 2
Stability of Budesonide solution formulations (inverted)
Recovery (%) of budesonide after storage at different times and
temperatures (average values referred to two tests)

| Can Type | | t = 2 months | t = 5 months |
|---|---|---|---|
| (cut edge can) | t = 0 | T = 50° C. | T = 50° C. |
| Anodized Aluminum | 99.18% | 96.64% | 76.94% |
| Lacquered Epoxy- | 99.52% | 97.28% | 71.03% |
| Phenol | | | |

t = time
T = temperature

### EXAMPLE 3

Two cans of anodized aluminum prepared as per example 2 were submitted to the determination of degradation products of the active ingredient.

It was noticed in this way that after a lag time of 6 months at 40° C. the degradation rate increases with the same pattern seen in the first example. The phenomenon does not occur if the cans are stored upright, because a direct contact between the solution and the rubbers is avoided. An explanation of the process is that peroxides may leach into the solution after a while, thus starting a radicalic degradation of Budesonide.

TABLE 3
Stability of Budesonide solution formulations (anodized
aluminum cut edge can)
Recovery (%) of degradation products desonide,
budesonide-21-aldehyde, budesonide-17-acid (related
substances) at different times, temperatures and
relative humidities (RH).

| | | 6 months | 6 months | 6 months |
|---|---|---|---|---|
| | | 40° C./ | 40° C./ | 30° C./ |
| | | 75% RH | 75% RH | 60% RH |
| TEST | START | Inverted | Upright | Inverted |
| Related substances (%): | | | | |
| desonide | 0.15 | 0.14 | 0.17 | <0.06 |
| bud-21-ald. | 0.16 | 2.96 | 1.19 | 0.98 |
| bud-17-ac. | — | 0.99 | 0.35 | 0.34 |
| unknowns | 0.27 | 0.49 | 0.37 | 0.18 |
| TOTAL | 0.58 | 4.58 | 2.08 | 1.50 |

### EXAMPLE 4

It has been observed that when the experiment is repeated changing only the finishing of the cans, (from cut edge to rolled neck; see FIG. 2) the stability of the solution is greatly increased due to two factors:

the different finishing of the can doesn't cause surface damages and/or cuts of the rubber gaskets, therefore avoiding the exposure of fresh surfaces not exposed during the ethanol extraction process;

the total gasket area exposed to the solution is much lower.

In conclusion, the combined action of ethanol extraction of the rubber gaskets of the valves and the finishing of the neck of the cans greatly improves the stability of solution formulations that otherwise may incur oxidative degradation triggered by the presence of peroxides in the above-mentioned gaskets.

TABLE 4
Stability of Budesonide solution formulations
(Epoxy-Phenol lacquered rolled neck can)
Recovery (%) of degradation products desonide, budesonide-21-
aldehyde, budesonide-17-acid (related substances) at different
times, temperatures and relative humidities (RH)

| | | 6 months | 12 months |
|---|---|---|---|
| | | 40° C./75% RH | 25° C./60% RH |
| TEST | START | Inverted | Inverted |
| Related substances | | | |
| (%): | | | |
| desonide | 0.14 | 0.23 | 0.16 |
| bud-21-ald. | 0.24 | 1.22 | 0.85 |
| bud-17-ac. | — | — | — |
| unknowns | 0.29 | 0.60 | 0.37 |
| TOTAL | 0.67 | 2.05 | 1.38 |

TABLE 5
Stability of budesonide solution formulations stored
in anodised aluminium rolled-in and rolled-over
cans in comparison with cut edge cans.
Recovery (%) of degradation products:
budesonide-21-aldehyde at different times and temperatures.

| | | | | 1 month |
|---|---|---|---|---|
| | | 1 month | 3 months | 40° C. |
| | | 50° C. | 50° C. | 75% R H |
| TEST | Start | Inverted | Inverted | Inverted |
| Related substances | % | % | % | % |
| cut edge | <0.02 | 0.58 | 1.76 | — |
| Bud-21-ald. - rolled in | <0.02 | 0.50 | 1.45 | — |
| rolled over | 0.08 | — | — | 0.23 |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A medicinal aerosol solution formulation product with improved chemical stability, comprising:
an aerosol canister equipped with a metering valve, said metering valve including a rubber valve gasket;
said aerosol canister containing a medicinal aerosol solution formulation comprising an active ingredient subject to a degradation by means of peroxides and/or other leachables, a hydrofluorocarbon propellant, a co-solvent, and optionally a low-volatility component; and
said aerosol canister having a rim with rounded edges adapted to prevent contact of a sharp edge with said rubber gasket.

2. The product of claim 1, wherein the canister has a rolled neck.

3. The product of claim 1, wherein the canister has a rolled-in rim.

4. The product of claim 1, wherein the canister has a partly rollover rim.

5. The product of claim 1, wherein the canister has a full rollover rim.

6. The product of claim 1, wherein the valve is washed before crimping of the valve upon the canister with a pharmaceutically acceptable solvent.

7. The product of claim 1, wherein the valve is washed before crimping of the valve upon the canister with ethanol.

8. The product of claim 1, wherein the active ingredient is a corticosteroid.

9. The product of claim 8, wherein the corticosteroid is a 20-ketosteroid.

10. The product of claim 9, wherein the 20-ketosteroid is selected from the group consisting of budesonide and its epimers, mometasone furoate, triamcinolone acetonide, butixocort and ciclesonide.

11. The product according to claim 1, wherein the low-volatility component is selected from the group consisting of glycerol, propylene glycol, polyethylene glycol and isopropyl myristate.

12. The product according to claim 1, wherein the co-solvent is ethanol.

13. The product according to 1, wherein the propellant is selected from HFA 227, HFA 134a and their mixtures.

14. The product according to claim 1, wherein part or all of the internal surfaces of said canister consists of stainless steel, anodized aluminum or are lined with an inert organic coating.

15. The product according to claim 14, wherein the inert organic coating is perfluoroalkoxyalkane, perfluoroalkoxyalkylene, perfluoroalkylenes such as polytetrafluoroethylene, epoxy-phenol resin or fluorinated-ethylene-propylene, polyether sulfone, or their combinations.

16. The product according to claim 1, wherein part or all of the internal surfaces consist of anodized aluminum.

17. A process for making a chemically stable aerosol solution formulation product containing an active ingredient subject to a degradation by means of peroxides or other leachables, comprising the steps of:
forming a rim on a canister having rounded edges;
filling the canister with a pressurized aerosol solution formulation, said formulation comprising an active ingredient subject to a degradation by means of peroxides or other leachables,
providing the canister with a valve having a rubber gasket as a component thereof, wherein the rounded edges of the canister prevent contact of a sharp edge with the rubber used as a valve gasket.

18. The process according to claim 17, wherein the rim of the canister is selected from the group consisting of a rolled neck, a rolled-in rim, a part rollover rim and a full rollover rim.

19. A process according to claim 17, further comprising the step of washing the valve with ethanol before attaching the valve to the canister.

20. The process according to claim 17, wherein part or all of the internal surfaces of said canister consists of stainless steel, anodized aluminum or are lined with an inert organic coating.

21. A process for the stabilization of an aerosol solution formulation containing an active ingredient subject to a degradation by means of peroxides or other leachables contained in a pressurized metered dose inhaler, comprising the steps of
providing a canister with a rim having rounded edges; and
washing a valve for use in connection with said canister with ethanol prior to connecting said valve to said canister.

22. The process according to claim 21, wherein said canister has a rolled neck.

23. The process according to claim 21, wherein said canister has a neck selected from the group consisting of a rolled-in rim, a part rollover rim and a full rollover rim.
